(19) United States
(12) Patent Application Publication
(10) Pub. No.: US 2025/0114501 A1
(43) Pub. Date: Apr. 10, 2025

(54) BREAST PUMP CONTROL METHOD, AND CIRCUIT CONTROL SYSTEM AND METHOD

(71) Applicant: Shenzhen TPH Technology Co.,Ltd., Shenzhen (CN)

(72) Inventor: Xianzi CHEN, Shenzhen (CN)

(21) Appl. No.: 18/984,989

(22) Filed: Dec. 17, 2024

Related U.S. Application Data
(63) Continuation of application No. PCT/CN2024/123726, filed on Oct. 9, 2024.

(30) Foreign Application Priority Data
Sep. 29, 2024 (CN) 202411374263.9

(51) Int. Cl. A61M 1/06

(52) U.S. Cl. CPC A61M 1/069

(57) ABSTRACT

A breast pump control method is provided. A breast shield is provided with a first cavity. The first cavity is allowed to fit and at least partially accommodate a breast to form a sealed area. A flexible component is provided. The flexible component is provided with a first magnetic component. At least a portion of the flexible component is communicated with the sealed area to form a portion of a wall of the sealed area. A second magnetic component is arranged opposite to the first magnetic component. The first magnetic component is driven by the second magnetic component to move to drive the flexible component to deform, such that a negative pressure is generated in the sealed area to drive the breast to express milk from the breast. A breast pump circuit control system and method are also provided.

### CROSS-REFERENCE TO RELATED APPLICATIONS

This application is a continuation of International Patent Application No. PCT/CN2024/123726, filed on Oct. 9, 2024, which claims the benefit of priority from Chinese Patent Application No. 202411374263.9, filed on Sep. 29, 2024. The content of the aforementioned application, including any intervening amendments made thereto, is incorporated herein by reference in its entirety.

### TECHNICAL FIELD

This application relates to breast pumps, and more particularly to a breast pump control method, and a circuit control system and method.

### BACKGROUND

Postpartum mothers usually return to work several weeks after giving birth. In order to provide breast milk to babies in a timely manner and relieve discomfort such as swelling and pain caused by failing to empty the breasts in time when breast milk is secreted relatively much, mothers can use breast pumps to pump breast milk and store the pumped milk properly.

In the prior art, breast pumps generally adopt a combination of a vacuum pump and a solenoid valve to pump breast milk. The milk pumping is achieved by controlling the vacuum pump, which will generate a large noise, thus greatly affecting the user's mood and causing poor comfort. In view of this, the present disclosure proposes a breast pump control method, and a circuit control system and method, so as to solve such problems.

### SUMMARY

An object of the disclosure is to provide a breast pump control method, and a circuit control system and method, so as to solve the problems in the prior art.

In order to achieve the above object, the following technical solutions are adopted.

In a first aspect, this application provides a breast pump control method, comprising:
step (1) providing a breast shield to form a first cavity, and allowing the first cavity to fit and at least partially accommodate a breast to form a sealed area;
step (2) providing a flexible component, wherein the flexible component is provided with a first magnetic component; and communicating at least a portion of the flexible component with the sealed area to form a portion of a wall of the sealed area; and
step (3) arranging a second magnetic component opposite to the first magnetic component; and driving, by the second magnetic component, the first magnetic component to move to drive the flexible component to deform, such that a negative pressure is generated in the sealed area to express milk from the breast.

In a second aspect, this application provides a breast pump circuit control system applied to the above breast pump control method for circuit control, comprising:
a microcontroller unit (MCU) control module;
an interactive control module; and
a voltage-and-current control module;
wherein the MCU control module is configured for signal reception and transmission;
interactive control module is configured to receive an operation signal from a user and transmit the operation signal to the MCU control module; and
the voltage-and-current control module is configured to receive a control signal transmitted by the MCU control module, control a voltage and a current passing through an electromagnetic coil to reach a desired suction force level, and transmit a suction level signal back to the MCU control module.

In a third aspect, this application provides a breast pump circuit control method for controlling above the breast pump circuit control system, comprising:
(1) determining, by the interactive control module, whether there is a button operation instruction; if yes, transmitting the button operation instruction to the MCU control module; otherwise, controlling a breast pump to automatically shut down;
(2) receiving and judging, by the MCU control module, the button operation instruction transmitted by the interactive control module followed by transmitting the button operation instruction to the voltage-and-current control module; and
(3) after receiving the button operation instruction, controlling, by the voltage-and-current control module, the voltage and the current passing through the electromagnetic coil, thereby controlling a magnitude of an electromagnetic force to generate the desired suction force level.

Compared to the prior art, the present disclosure has the following beneficial effects.

The present disclosure adopts the second magnetic component to control the movement of the first magnetic component to drive the flexible component to deform, such that the negative pressure is generated in the sealed area to express milk from the breast. Compared with traditional vacuum pumps and solenoid valves, this eliminates the source of noise to significantly reduce the noise or even reach “zero” noise, thereby eliminating the impact of noise on the user's emotion and improving comfort.

### BRIEF DESCRIPTION OF THE DRAWINGS

In order to illustrate the technical solutions in the embodiments of the present disclosure or in the prior art more clearly, the drawings needed in the description of embodiments or the prior art will be briefly introduced below. Obviously, for those of ordinary skill in the art, other drawings can be obtained based on these drawings without exerting creative efforts.

FIG. 1 is a structural diagram of a breast pump in accordance with an embodiment of the present disclosure;

FIG. 2 is an exploded view of the breast pump in accordance with an embodiment of the present disclosure;

FIG. 3 schematically shows assembly structure of the breast pump in accordance with an embodiment of the present disclosure;

FIG. 4 is a cross-sectional view of the breast pump in accordance with an embodiment of the present disclosure;

FIG. 5 is an enlarged view of part A in FIG. 4;

FIG. 6 is a structural diagram of a connector of the breast pump in accordance with an embodiment of the present disclosure;

FIG. 7 is a structural diagram of a cover of the breast pump in accordance with an embodiment of the present disclosure;

FIG. 8 is a structural diagram of a flexible component of the breast pump in accordance with an embodiment of the present disclosure;

FIG. 9 is a structural diagram of an interior of the flexible component in accordance with an embodiment of the present disclosure;

FIG. 10 is a block diagram of a wearable breast pump control system in accordance with an embodiment of the present disclosure; and

FIG. 11 is a flow chart of a wearable breast pump control method in accordance with an embodiment of the present disclosure.

In the drawings: 1. first housing; 11. breast shield; 111. first cavity; 112. second cavity; 12. cover; 121. mounting portion; 122. convex ring; 123. assembly groove; 124. through hole; 125. air groove; 126. first quick-release structure; 127. liquid outlet; 2. second housing; 21. first half-housing; 211. button; 212. charging port; 22. second half-housing; 221. second quick-release structure; 3. connector; 31. mounting sleeve; 311. limiting component; 32. first connecting tube; 33. second connecting tube; 34. mounting structure; 341. first clamping portion; 4. flexible component; 4a. assembly portion; 4b. deformation portion; 41. first magnetic component; 42. second clamping portion; 43. annular groove; 44. protrusion; 45. third magnetic component; 46. valley; 47. air hole; 5. second magnetic component; 6. control panel; 7. power supply device; and 8. one-way valve.

The object, functional features and advantages of the present disclosure will be further described below with reference to the embodiments and the accompanying drawings.

### DETAILED DESCRIPTION OF EMBODIMENTS

The technical solutions of the present disclosure will be clearly and completely described below in conjunction with the accompanying drawings and embodiments.

Obviously, described below are only some embodiments of the present disclosure, instead of all embodiments of the present disclosure. Based on the embodiments in the present disclosure, all other embodiments obtained by those of ordinary skill in the art without making creative efforts shall fall within the scope of the present disclosure.

It should be noted that all directional indications (such as up, down, left, right, front, back . . . ) in the description of the embodiments are merely intended to explain a relative positional relationship, movement, etc. between components in a specific posture (as shown in the accompanying drawings). When the specific posture changes, the directional indication changes accordingly.

In addition, descriptions involving “first”, “second”, etc. in this application are only descriptive, and cannot be understood as indicating or implying relative importance or implicitly indicating the number of indicated technical features. Therefore, features defined as “first” and “second” can explicitly or implicitly include at least one of the features. In addition, “and/or” in the entire specification includes three solutions. For example, A and/or B includes technical solution A, technical solution B, and a combination of A and B. Moreover, technical solutions in the embodiments can be combined with each other, but must be based on what can be achieved by those of ordinary skill in the art. When the combination of technical solutions appears to be contradictory or cannot be realized, it should be deemed that such combination of technical solutions does not exist and is not within the scope of the present disclosure defined by the appended claims.

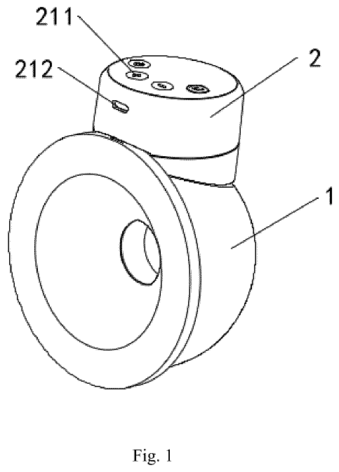

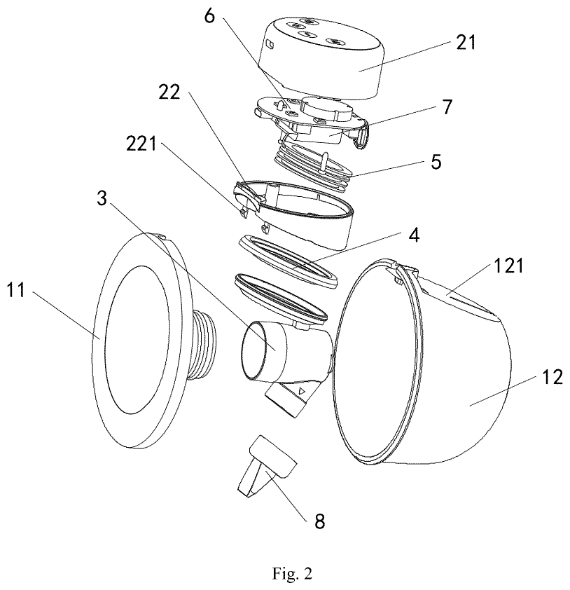

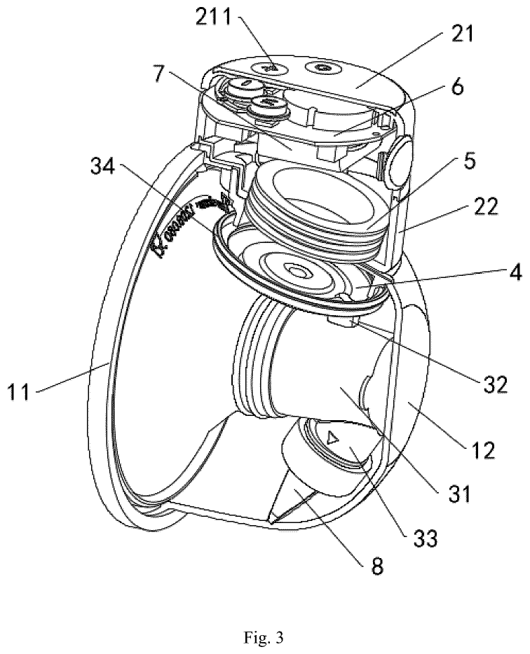

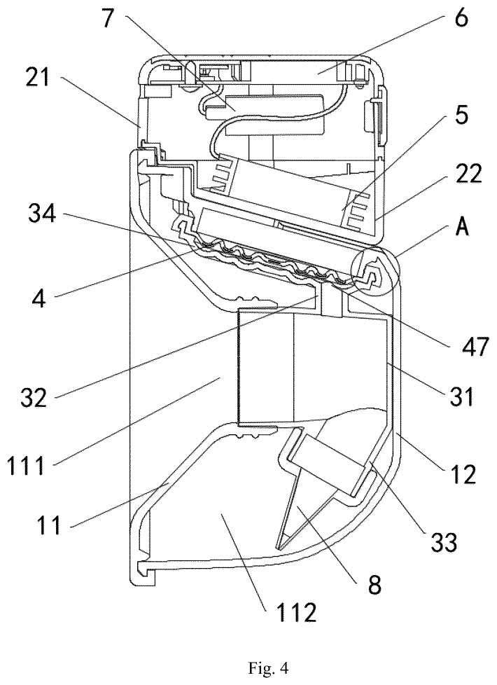

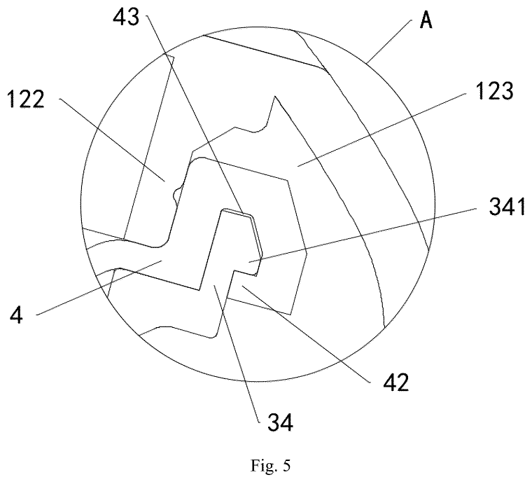

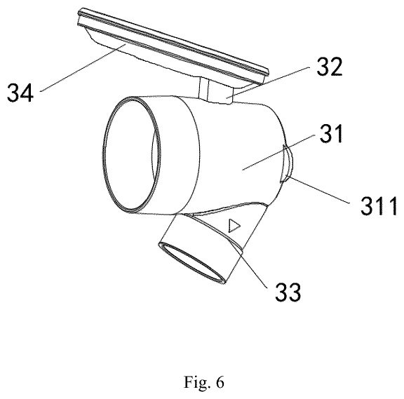

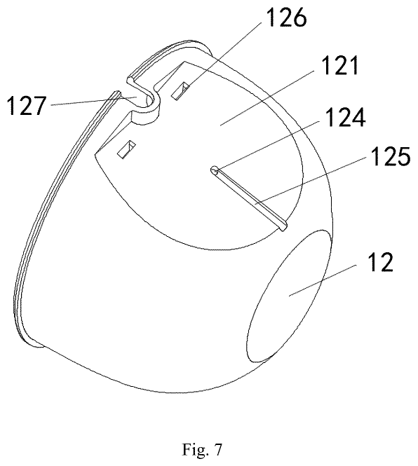

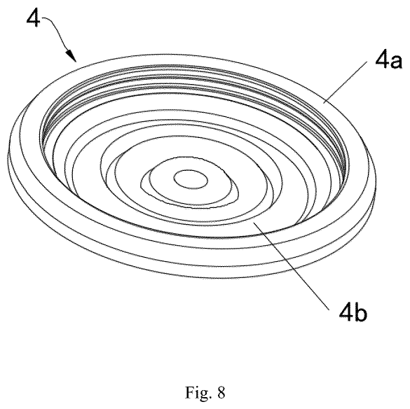

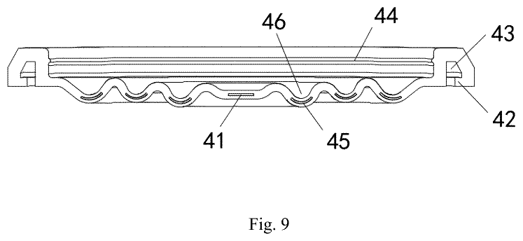

As shown in FIGS. 1-9, a breast pump driven by magnetic force to generate a negative pressure is provided, which includes a breast shield 11, a flexible component 4 and a second magnetic component 5.

The breast shield 11 is provided with a first cavity 111. The first cavity 111 is in gapless fit with and at least partially accommodate the breast to form a sealed area.

A first magnetic component 41 is provided on the flexible component 4. At least a portion of the flexible component 4 is communicated with the sealed area to form a portion of a wall of the sealed area.

The first magnetic component 41 is arranged opposite to the second magnetic component 5. The second magnetic component 5 is configured to magnetically drive the first magnetic component 41 to move to drive the flexible component 4 to deform, such that a negative pressure is generated in the sealed area.

In this embodiment, an inner wall of the breast shield 11 can fit a user's breast. In this case, after the user's breast is fitted with the inner wall of the breast shield 11, the sealed area enclosed by the breast shield 11 and the flexible component 4 is in a sealed state.

The first magnetic component 41 is arranged opposite to the second magnetic component 5, and positions of the first magnetic component 41 and the second magnetic component 5 can be relatively exchanged. It should be noted that the magnetic components herein do not only refer to components having magnetic force, but also any material that can be guided by magnetic force to move.

In an embodiment, the first magnetic component 41 is, but not limited to, a permanent magnet. The first magnetic component 41 can be in an annular shape, a block shape or a strip shape. The second magnetic component 5 is an electromagnetic coil, and is in an annular shape, a block shape or a strip shape.

When operating, the inner wall of the breast shield 11 is configured to fit the user's breast. The second magnetic component 5 is started, such that a magnetic force is generated by the second magnetic component 5 to drive the first magnetic component 41 to move. In other words, when a magnetic pole of the second magnetic component 5 is different from that of the first magnetic component 41, an attraction force is generated between the first magnetic component 41 and the second magnetic component 5, such that the flexible component 4 is driven to deform under an action of the attraction force, so as to generate the negative pressure inside the sealed area. At this time, with the cooperation of the breast shield 11, pressure will be exerted on the user's breast to express the breast milk. When the magnetic pole of the second magnetic component 5 is the same as that of the first magnetic component 41, a repulsion force is generated between the first magnetic component 41 and the second magnetic component 5, such that the flexible component 4 is driven to reset, and the negative pressure inside the sealed area disappears.

In the present disclosure, electromagnetic milk pumping is adopted to replace the vacuum pump plus electromagnetic valve milk pumping in the prior art. This not only significantly reduces the weight of the breast pump, making it easier to carry, but also eliminates the vacuum pump and electromagnetic valve to significantly reduce the noise or even reach the “zero” noise, thereby eliminating the impact of noise on the user's mood and improving the comfort.

In addition, components such as the vacuum pump and the electromagnetic valve are canceled, and the magnetic attraction is adopted instead, which reduces the assembly components (such as the vacuum pump, solenoid valve and sealing ring), thereby effectively reducing costs.

In an embodiment, at least one third magnetic component 45 is arranged inside the flexible component 4, and the at least one third magnetic component 45 is located around a periphery of the first magnetic component 41.

In this embodiment, arranging the at least one third magnetic component 45 embedded inside the flexible component 4 can further improve the magnetic property, thereby improving efficiency and reducing energy loss. The at least one third magnetic component 45 is a neodymium magnet.

The at least one third magnetic component 45 is in an annular shape. In an embodiment, the at least one third magnetic component 45 is configured as two or more third magnetic components 45 that are arranged in an ascending order in terms of diameter along a radius direction of the flexible component 4 from the center to the outside.

In an embodiment, the sealed area is communicated with a milk collecting component configured to collect and store breast milk.

In this embodiment, any container that can collect and store the breast milk can be considered as a milk collecting component, such as a feeding bottle and a bowl-shaped container.

In an embodiment, the milk collecting component includes a cover 12. The cover 12 and the breast shield 11 configured to enclose a first housing 1. An interior of the first housing 1 is provided with a second cavity 112. A converging end of the breast shield 11 extends into the second cavity 112.

In this embodiment, the breast shield 11 is sealedly connected to the cover 12. The cover 12 is in a shape similar to a hemisphere. An edge of an opening of the cover 12 is provided with a liquid outlet 127 that is configured to pour out the breast milk after the pumping is completed.

In an embodiment, the breast pump further includes a second housing 2. The second magnetic component 5 is fixedly arranged at the second housing 2, and can be alternately switched between the S-pole and the N-pole.

In this embodiment, the second magnetic component 5 is allowed to be alternately switched between the S-pole and the N-pole, such that the flexible component 4 is driven to achieve the reciprocating motion between deformation and resetting, thereby effectively achieving the stable “sucking” and “releasing” functions. In this way, the breast pump can achieve a continuous and stable milk pumping process.

In an embodiment, the first magnetic component 41 is configured as an S-pole. When the second magnetic component 5 is configured as an N-pole, an attraction force is generated between the first magnetic component 41 and the second magnetic component 5, such that the flexible component 4 is driven to deform, and a negative pressure is generated inside the sealed area, so as to achieve milk pumping operation. When the second magnetic component 5 is configured as an S-pole, a repulsion force is generated between the first magnetic component 41 and the second magnetic component 5, such that the flexible component 4 is pulled to reset until the negative pressure inside the sealed area disappears.

In the prior art, the negative pressure created by the vacuum pump has a delay in conduction, and the efficiency can only be improved by relying on the original material rebound performance. The air in the housing and the flexible component must be evacuated to generate negative pressure on the user's breast. In contrast, the magnetic attraction method adopted herein can instantly change the direction of the magnetic field and the magnitude of the magnetic field strength. The flexible component 4 can be driven by the repulsion force to quickly reset, which can reduce the delay and greatly improve the milk pumping efficiency.

In an embodiment, the breast pump further includes a connector 3. The breast shield 11 is connected to the flexible component 4 through the connector 3 to form the sealed area. The connector 3 has a hollow structure. One end of the connector 3 is provided with an opening, and is connected to the converging end of the breast shield 11.

In this embodiment, the connector 3 is, but not limited to, a three-way valve. The breast shield 11 and the flexible component 4 are each independently detachably and sealedly connected to the connector 3.

In an embodiment, the connector 3 is communicated with the milk collecting component, and a one-way valve 8 is provided between the connector 3 and the milk collecting component.

In this embodiment, a one-way valve 8 is provided, so that when the negative pressure is generated in the sealed area, the breast milk in the milk collecting component can be prevented from flowing back into the sealed area. The one-way valve 8 is a duckbill valve. Specifically, when the negative pressure is generated in the sealed area, the air pressure inside the sealed area is lower than the external air pressure, which will cause the one-way valve 8 to deform and close. Moreover, the closing of the one-way valve 8 can also prevent gas leakage and increase air tightness, thereby improving the milk pumping effect. When the negative pressure disappears, the one-way valve 8 will reset and open, and the squeezed breast milk will flow into the cover 12 through the one-way valve 8.

In an embodiment, the connector 3 is provided with a mounting structure 34. An interior of the mounting structure 34 is communicated with the connector 3, and the flexible component 4 is arranged in the mounting structure 34.

In this embodiment, the mounting structure 34 is a suction bowl. The mounting structure 34 has various shaped structures, such as a circular structure, a square structure or a plane structure. Any structure that can achieve the fixation of the flexible component 4 can be configured as the mounting structure 34 in this disclosure. An edge of the flexible component 4 is connected to an edge of the mounting structure 34 by means of sealing or interference connection, so that the interior of the mounting structure 34 is in a relatively sealed state. In an embodiment, the flexible component 4 is in fit with the mounting structure 34 in terms of shape.

A bottom of the mounting structure 34 is provided with an air hole 47 that is configured such that the interior of the mounting structure 34 is communicated with the connector 3.

In an embodiment, the mounting structure 34 is configured to fit the flexible component 4.

In this embodiment, the mounting structure 34 and the flexible component 4 are arranged to fit each other, so that there is no gap between the mounting structure 34 and the flexible component 4. When performing breast milk pumping, if the breast milk is accidentally sucked into the mounting structure 34, the rebound of the flexible component 4 will promptly discharge the breast milk from the mounting structure 34. In this way, the breast milk will not remain in the mounting structure 34.

In an embodiment, an inner wall of the cover 12 is provided with a convex ring 122. The cover 12 is connected to the mounting structure 34 through the convex ring 122. The convex ring 122 extends from an opening of the mounting structure 34 to the interior of the mounting structure 34. The flexible component 4 is configured to be clamped by the convex ring 122 and the mounting structure 34.

In this embodiment, the convex ring 122 is shapely consistent with a bowl mouth of the mounting structure 34. One side of the convex ring 122 and the inner wall of the cover 12 are configured as an assembly groove 123. After the assembly between the flexible component 4 and the mounting structure 34 is completed, the mounting structure 34 mounted with the flexible component 4 is inserted into the assembly groove 123. At this time, the convex ring 122 is inserted into the mounting structure 34 to complete the assembly, which also has a positioning function.

In addition, in a case where a distance between an outer wall of the convex ring 122 and an inner wall of the mounting structure 34 is greater than the thickness of the flexible component 4, if the flexible component 4 is deformed, the convex ring 122 can enhance the connection strength between the flexible component 4 and the mounting structure 34 to prevent the sealing performance of the connection from being reduced due to the deformation of the flexible component 4. In a case where the distance between the outer wall of the convex ring 122 and the inner wall of the mounting structure 34 is slightly less than the thickness of the flexible component 4, in addition to increasing the connection strength between the flexible component 4 and the mounting structure 34, the flexible component 4 will be clamped by the convex ring 122 and the mounting structure 34 to form an interference fit, so that the flexible component 4 tightly fit the mounting structure 34. In this way, the sealing effect can be further improved.

In an embodiment, the cover 12 is provided with a mounting portion 121 that is configured as a slot or a magnetic attraction structure. The cover 12 is detachably connected to the second housing 2 through the mounting portion 121.

In an embodiment, a through hole 124 is provided on a surface of the cover 12, and is communicated with the sealed area formed by the cover 12 and the flexible component 4.

In this embodiment, the through hole 124 is configured such that the pressure in the sealed area formed by the cover 12 and the flexible component 4 is consistent with the atmospheric pressure, thereby allowing the flexible component 4 to be reset.

In an embodiment, the surface of the cover 12 is provided with an air groove 125. The air groove 125 is communicated with the through hole 124.

In this embodiment, when the through hole 124 is arranged on the mounting portion 121, the interior of the cover 12 can still be communicated with the exterior through the air groove 125, so that the pressure in the sealed area is consistent with the atmospheric pressure without being affected by the second housing 2.

In an embodiment, the second magnetic component 5 is configured to be controlled by a control mechanism that is arranged in the second housing 2.

In this embodiment, the pole of the second magnetic component 5 can be controlled by the control mechanism, so as to achieve the transformation of the second magnetic component 5 between the S-pole and the N-pole.

In an embodiment, the control mechanism includes a control panel 6 and a power supply device 7. The control panel 6 is electrically connected to the second magnetic component 5. The power supply device 7 is electrically connected to the control panel 6.

In this embodiment, the power supply device 7 is a battery arranged in one. In other embodiments, the number of the batteries can be two or more. The power supply device 7 is configured to supply power to the second magnetic component 5 and the control panel 6.

Specifically, when the second magnetic component 5 is powered, the current will cause the second magnetic component 5 to generate a magnetic field, thereby generating a pole. The direction and magnitude of the current can be controlled by the control panel 6, so as to control the pole conversion of the second magnetic component 5 and the strength of the magnetic field.

More specifically, the first magnetic component 41 is configured as the S-pole. In a case where the current is configured such that the second magnetic component 5 is the N-pole, the attraction force is generated between the first magnetic component 41 and the second magnetic component 5, such that the negative pressure is generated inside the sealed area, so as to realize the milk pumping operation. In a case where the current is configured such that the second magnetic component 5 is the S-pole, the repulsion force is generated between the first magnetic component 41 and the second magnetic component 5, such that flexible component 4 is pulled to be away from the second housing 2 for resetting, and the negative pressure inside the cavity disappears.

In this way, by precisely controlling the magnitude of the current, the magnitude of the magnetic field strength and the magnitude of the suction force can be precisely controlled, thereby achieving the adjustment of the negative pressure and effectively improving the comfort level.

In an embodiment, the second housing 2 includes a first half-housing 21 and a second half-housing 22. The first half-housing 21 is detachably connected to the second half-housing 22. A lower end of the second half-housing 22 is provided with a second quick-release structure 221, and is detachably mounted on the mounting portion 121 through the second quick-release structure 221.

In this embodiment, the first half-housing 21 is detachably connected to the second half-housing 22, and the lower end of the second half-housing 22 is detachably mounted on the mounting portion 121, which facilitates the assembly and disassembly of the breast pump.

The mounting portion 121 is provided with a first quick-release structure 126 in correspondence with the second quick-release structure 221. When the first quick-release structure 126 is a snap-fit hole, the second quick-release structure 221 can be a snap buckle. In addition, both the first quick-release structure 126 and the second quick-release structure 221 can be magnetic attraction components.

In an embodiment, the first half-housing 21 is provided with a plurality of buttons 211.

In this embodiment, different buttons 211 are set to send different control instructions to the controller to achieve different functions.

Specifically, the plurality of buttons 211 include a power button, a level adjustment button, a pause button and a mode switch button. The power button is configured to control the start and stop of the breast pump. The level adjustment button includes a “+” button for shift-up and a “−” button for shift-down. The pause button is configured to control the breast pump to pause. The breast pump in this application has a massage mode, a milk pumping mode, a stimulation mode and an automatic mode, and the mode switch button is configured to switch the breast pump among the four modes.

In an embodiment, the cover 12 is transparent. An outer surface of the cover 12 is provided with a plurality of scale lines.

In this embodiment, the cover 12 is designed to be transparent, so that the breast milk collection status and internal cleanliness can be observed in real time, which facilitates the timely cleaning and preventing the bacterial growth.

In addition, the plurality of scale lines are arranged at the outer surface of the cover 12, and are different in terms of marking, so as to measure the amount of pumped breast milk.

In this embodiment, the first half-housing 21 is provided with a display screen that is electrically connected to the control panel 6.

In this embodiment, the display screen can display pressure, power, charging status, time and level information.

In an embodiment, the connector 3 includes a mounting sleeve 31, a first connecting tube 32 and a second connecting tube 33. One end of the mounting sleeve 31 is provided with an opening, which is detachably connected to the converging end of the breast shield 11. A first side of the mounting sleeve 31 is connected to the air hole 47 through the first connecting tube 32, and a second side of the mounting sleeve 31 is connected to a first end of the second connecting tube 33 to be communicated with the cover 12. The one-way valve 8 is sleevedly provided at a second end of the second connecting tube 33 away from the mounting sleeve 31. The first connecting tube 32 is located above the second connecting tube 33.

In this embodiment, the opening end of the mounting sleeve 31 is connected to the converging end of the breast shield 11 by means of sealing or interference connection, thereby achieving a relative sealing connection. In this embodiment, a cross-section of the mounting sleeve 31 is in a circular shape, which can be in polygonal or other irregular shapes in other embodiments. The second connecting tube 33 is provided so that the breast milk in the mounting sleeve 31 can flow into the cover 12 through the second connecting tube 33 for storage.

In an embodiment, the connector 3 is provided with a limiting component 311. The limiting component 311 is configured to prevent the connector 3 form moving relative to the milk collecting portion.

In this embodiment, the limiting component 311 is in a semicircular-sheet shape, and is configured as at least two limiting components 311 that are respectively located on both sides of the connector 3. By virtue of the limiting component 311, the connector 3 can be limited and positioned when assembled to the cover 12.

In an embodiment, an outer edge of the opening of the mounting structure 34 is provided with a first clamping portion 341 in a hook shape, a bowl wall of the mounting structure 34 and the first clamping portion 341 are configured to together form a hook structure. An upper end of the hook structure is arranged to be inclined toward the bottom of the cover 12. The outer edge of the flexible component 4 is configured to bend downward to form an annular groove 43. The outer edge of the flexible component 4 is provided with a second clamping portion 42 in a hook shape. The second clamping portion 42 is configured to extend to an interior of the annular groove 43. When the first clamping portion 341 is inserted into the annular groove 43, the first clamping portion 341 is in snap-fit with the second clamping portion 42.

In this embodiment, the second clamping portion 42 is arranged at the opening of the annular groove 43 to form a converging shape. During mounting, once the first clamping portion 341 is inserted into the annular groove 43, the second clamping portion 42 can be in snap-fit with the first clamping portion 341, and it is not easy to separate. Under the action of the elastic force of the flexible component 4 and the hook structure inclinedly arranged, the flexible component 4 is tightened, so that the mounting structure 34 can be tightly connected to the flexible component 4, thereby achieving a better sealing effect.

A flexible membrane driven by magnetic force to generate negative pressure is provided, which is the flexible component 4 of the above breast pump. The flexible membrane includes an assembly portion 4a and a deformation portion 4b. The assembly portion 4a is configured to position and fix the membrane body of the flexible membrane. The deformation portion 4b is integrally formed with the assembly portion 4a. The deformation portion 4b is sealedly connected to the assembly portion 4a. The assembly portion 4a is arranged at an edge of the deformation portion 4b.

In this embodiment, the deformation portion 4b is in a circular-sheet shape. In some embodiments, the deformation portion 4b can be in a polygon shape or other irregular shapes. The assembly portion 4a is in an annular shape that is in fit with the shape of deformation portion 4b. The deformation portion 4b and the assembly portion 4a are integrally formed into a bowl-shaped structure by means of injection molding. The first magnetic component 41 is in a block shape, and is located at a center of the deformation portion 4b. The assembly portion 4a and the deformation portion 4b are made of latex. An edge of the assembly portion 4a is provided with a groove structure, i.e., the annular groove 43. The membrane body is mounted on the mounting structure 34 through the groove structure, so that the flexible membrane can be quickly buckled with the suction bowl, resulting in a convenient assembly. The groove structure is located outside the deformation portion 4b, and has an opening facing downward.

The second clamping portion 42 is arranged on the inner wall of the opening of the annular groove 43 and protrudes toward the deformation portion 4b. The second clamping portion 42 is located on a side wall of the annular groove 43 away from the deformation portion 4b.

The first magnetic component 41 is embedded in the deformation portion 4b, so that the deformation portion 4b can be driven by magnetic force to deform, without the need for a vacuum pump or mechanical structure drive, which prevents the generation of noise from the source and does not affect the user's emotion.

In an embodiment, the at least one third magnetic component 45 is embedded in the deformation portion 4b, and is located at the periphery of the first magnetic component 41.

In this embodiment, the at least one third magnetic component 45 is configured as a plurality of third magnetic components 45 that are arranged in ascending order in terms of diameter along a radius direction of the deformation portion 4b from the inside to the outside.

In an embodiment, a cross-section of the deformation portion 4b is in a wave shape.

In this embodiment, the resilience performance of the deformation portion 4b can be increased by setting the deformation portion 4b in a wave shape, where the at least one third magnetic component 45 is arranged at a valley 46 of the deformation portion 4b. In an embodiment, a cross-section of the at least one third magnetic component 45 is in an arc shape, so that the at least one third magnetic component 45 is in fit with the valley 46 of the deformation portion 4b.

A molding process of the flexible membrane (i.e., flexible component 4) is provided herein.

First, the flexible membrane is integrally formed by means of injection molding.

Then, the first magnetic component 41 and the at least one third magnetic component 45 are embedded in the deformation portion 4b. The first magnetic component 41 and the at least one third magnetic component 45 are formed at a diaphragm of the suction bowl by means of overmolding. At this time, the neodymium alloy at the flexible membrane does not have magnetism. After the overmolding is completed, the flexible membrane (with the first magnetic component 41 and the at least one third magnetic component 45 arranged therein) is placed at a fixed position in a jig without moving freely, and transferred into a magnetizing machine for magnetization, so as to obtain a magnetized flexible membrane.

The specific steps are as follows.

(S1) A surface of a neodymium alloy (unmagnetized) is cleaned to remove oil and dust.

(S2) A treatment agent for bonding metal with silicone is applied to the surface of the neodymium alloy for silicone bonding, and then dried according to the instructions of the treatment agent.

(S3) The neodymium alloy is put into a mold to produce a silicone base by means of overmolding.

(S4) The silicone base is cleaned to remove oil and dust on the surface.

(S5) The silicone base is applied with the treatment agent and dried according to the instructions of the treatment agent.

(S6) The silicone base is put into the mold to produce a semi-finished flexible membrane (with the neodymium alloy arranged therein) by means of overmolding.

(S7) The semi-finished flexible membrane (with the neodymium alloy arranged therein) is put into a magnetizing fixture in order so that the semi-finished flexible membrane cannot move during the magnetizing process, and then put into the magnetizing machine for magnetization to obtain the magnetic flexible membrane.

In an embodiment, the flexible component 4 is provided with a protrusion 44 that is located on a side wall of the assembly portion 4a close to the deformation portion 4b and away from the annular groove 43.

In this embodiment, after the assembly between the convex ring 122 and the suction bowl is completed, an interference fit can also be formed between the protrusion 44 and the side wall of the convex ring 122, thereby improving the sealing performance of the connection.

In an embodiment, the first magnetic component 41 and the at least one third magnetic component 45 are both neodymium magnets.

In this embodiment, the neodymium magnets are obtained by magnetizing the neodymium alloy.

A magnetic drive system is also provided, which is composed of the components of the above breast pump driven by the magnetic force to generate negative pressure, including the flexible component 4, the mounting structure 34, the first magnetic component 41 and the second magnetic component 5.

The flexible component 4 is arranged on the mounting structure 34, and is sealedly connected to the mounting structure 34. The mounting structure 34 is provided with the air hole 47.

The first magnetic component 41 is arranged in the flexible component 4.

The second magnetic component 5 is arranged opposite to the first magnetic component 41, and is configured to be alternately switched between the S-pole and the N-pole.

In the present disclosure, electromagnetic milk pumping is adopted to replace the vacuum pump plus electromagnetic valve milk pumping in the prior art. The second magnetic component alternates between the S-pole and N-pole to drive the flexible component to deform, thus generating negative pressure for milk pumping. The cancellation of the vacuum pump and the solenoid valve eliminates the source of noise to significantly reduce the noise or even reach “zero” noise, thereby eliminating the impact of noise on the user's mood and improving comfort.

In an embodiment, the magnetic force of the first magnetic component 41 and the second magnetic component 5 (i.e., the electromagnetic coil) can be calculated according to the design requirements as follows:

$$P = F / S , F = P * S . \qquad ( 1 )$$

In the Equation (1), P is pressure, F is force, and S is force area.

From the conversion between air pressure and force, it can be known that 1 Pa=1 N/m², and 1 mmHg=133.3223684 Pascals (Pa).

On this basis, after the area of the mounting structure 34 is calculated, the force required to pull the flexible component 4 upward can be obtained through the Equation (1).

A Maxwell electromagnetic attraction force F can be calculated by Equation (2):

$$F = {B}^{2} * S / 2 * {μ}^{0} = {φ}^{2} / 2 * {μ}^{0} * S . \qquad ( 2 )$$

In the Equation (2), B is a magnetic induction intensity (T), S is a cross-sectional area of a magnetic circuit (m²), φ is a magnetic flux passing through the cross-section (Wb), and μ⁰ is a vacuum magnetic permeability (4π*10⁻⁷ Wb/A·m).

Excluding the leakage magnetic loss and the connection air gap loss, only the deformation active travel of the mounting structure 34 is considered as a main air gap, the magnetic induction intensity B at the built-in magnetic component of the mounting structure 34 is calculated through Equation (3):

$$B = N * U * {μ}^{0} / R * g = N * I * {μ}^{0} / g \qquad ( 3 )$$

In the Equation (3), N is the number of coil turns; I is a current intensity (A); U is a power supply voltage (V); R is a winding coil resistance (Ω); and g is a width of the air gap (m) (i.e., a distance from the built-in magnetic component of the mounting structure 34 to the cross-section of the coil).

The Equation (3) is substituted into the Equation (2), so as to calculate the electromagnetic attraction force F through Equation (4):

$$F = {( N * I )}^{2} * {μ}^{0} * S / 2 * {g}^{2} \qquad ( 4 )$$

The area of the first magnetic component 41 can be determined through the above design. The cross-sectional area S of the magnetic circuit of the coil can be calculated in the same way. Moreover, the distance g from the first magnetic component 41 to the cross-section of the second magnetic component 5 can also be calculated. The number of coil turns N and the current intensity I can be adopted as debugging values. N*I is proportional to F. When F is greater than the upward pulling force of the flexible component 4, the required negative pressure can be met.

A breast pump control method for controlling the above breast pump is provided, including the following steps.

(S1) A breast shield 11 is provided with a first cavity. The first cavity is allowed to fit and at least partially accommodate a breast to form a sealed area.

(S2) A flexible component 4 is provided. The flexible component 4 is provided with a first magnetic component 41. At least a portion of the flexible component 4 is communicated with the sealed area to form a portion of a wall of the sealed area.

(S3) A second magnetic component 5 is arranged opposite to the first magnetic component 41. The first magnetic component 41 is driven by the second magnetic component 5 to move to drive the flexible component 4 to deform, such that a negative pressure is generated in the sealed area to express milk from the breast.

In this embodiment, electromagnetic milk pumping is adopted to replace the vacuum pump plus electromagnetic valve milk pumping in the prior art. This significantly reduces the weight of the breast pump, making it easier to carry. Moreover, the second magnetic component 5 is adopted to drive the first magnetic component 41 to move, thus driving the flexible component 4 to deform, instead of using the vacuum pump and electromagnetic valve. In this way, the negative pressure is generated in the sealed area to drive the breast to express milk. This eliminates the source of noise to significantly reduce the noise or even reach “zero” noise, thereby eliminating the impact of noise on the user's mood and improving comfort.

In an embodiment, the step (S3) further includes the following step.

The second magnetic component is controlled to switch between a S-pole and an N-pole, such that the flexible component 4 is driven by the first magnetic component 41 to perform reciprocating motion.

In an embodiment, the step (S1) further includes the following steps.

A connector 3 is connected to the breast shield 11. The connector 3 has a hollow structure. An end of the connector is provided with an opening, and is connected to a converging end of the breast shield 11.

A suction bowl is arranged at the connector 3. An interior of the suction bowl is communicated with the connector 3.

The flexible component 4 is arranged in the suction bowl, such that the breast shield 11 and the flexible component 4 form the sealed area through the connector 3. In an embodiment, the step (S3) further includes the following steps.

The second magnetic component 5 is fixed in the second housing 2. The second magnetic component 5 is connected to a control mechanism, where the control mechanism is arranged in the second housing 2.

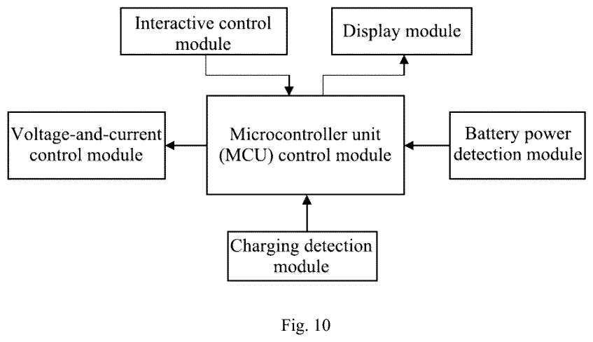

As shown in FIG. 10, a breast pump circuit control system applied to the above breast pump control method is provided, including a microcontroller unit (MCU) control module, an interactive control module and a voltage-and-current control module.

The MCU control module is configured for signal reception and transmission.

The interactive control module is configured to receive an operation signal from a user and transmit the operation signal to the MCU control module.

The voltage-and-current control module is configured to receive a control signal transmitted by the MCU control module, control a voltage and a current passing through an electromagnetic coil to reach a desired suction force level, and transmit a suction level signal back to the MCU control module.

In this embodiment, after the user operates one of the plurality of buttons 211, the interactive control module will send a corresponding button signal to the MCU control module. The MCU control module is configured to receive the signal transmitted by the interactive control module and transmit a control signal to the voltage-and-current control module. After receiving the control signal, the voltage-and-current control module controls the voltage and current of the electromagnetic coil according to the instruction of the signal, thereby controlling the magnitude of the electromagnetic force. In this way, the deformation degree of the flexible component 4 can be controlled, so as to generate suction forces with different strengths.

In an embodiment, the circuit control system further includes a power detection module configured to monitor a power level of a power supply device 7 in real time and transmit the power level to the MCU control module.

In this embodiment, the power detection module can monitor the power level of the battery inside the breast pump in real time and transmit monitoring data to the MCU control module.

In an embodiment, the circuit control system further includes a charging detection module configured to detect an external charger and feed a detection result back to the MCU control module.

In this embodiment, in a case where the battery of the breast pump is out of power, if the battery is charged, the charging detection module will perform charger detection and monitor the charging status in real time.

In an embodiment, the circuit control system further includes a display module. The MCU control module is configured to receive signals from the voltage-and-current control module, the power detection module and the charging detection module, and transmit the signals to the display module, and the display module is configured to receive and display the signals transmitted by the MCU control module.

In this embodiment, after the voltage-and-current control module, the power detection module and the charging detection module transmit signals to the MCU control module, the signals are transmitted by the MCU control module to the display module, and the display module can display the data information on a display screen.

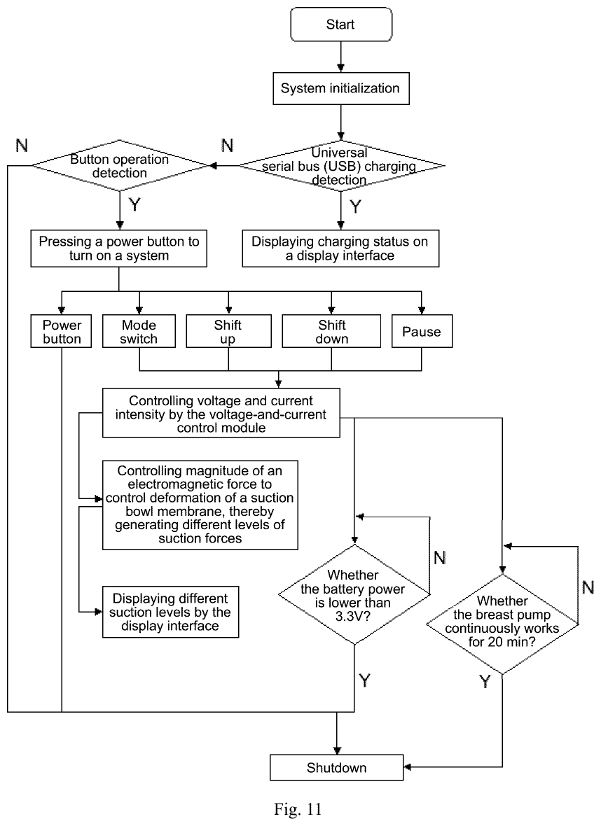

As shown in FIG. 11, a breast pump circuit control method for controlling the above breast pump circuit control system is provided, which includes the following steps.

(S1) The circuit control system is turned on and initialized.

(S2) Whether an external charger is connected is determined by the charging detection module. If yes, a charging status signal is transmitted to the display module for display.

(S3) Whether there is a button operation instruction is determined by the interactive control module. If yes, the button operation instruction is transmitted to the MCU control module, otherwise, the breast pump is controlled to automatically shut down.

(S4) The button operation instruction transmitted by the interactive control module is received and judged by the MCU control module, and then transmitted by the MCU control module to the voltage-and-current control module.

(S5) After receiving the button operation instruction, the voltage-and-current control module controls the voltage and current passing through the electromagnetic coil to control the magnitude of the electromagnetic force to generate the desired suction force level.)

In this embodiment, before the operation, the system of the breast pump needs to be initialized. After the initialization is completed, one of the plurality of buttons 211 is operated by the user. After the interactive control module determines that there is a button operation instruction, a power button is pressed to turn on the system. In this way, the it can be prevented that the breast pump is started due to accidental touch. After the system is turned on, the plurality of buttons 211 can be controlled. The instruction corresponding to the plurality of buttons 211 is transmitted by the interactive control module to the MCU control module, and then transmitted by the MCU control module to the voltage-and-current control module. The voltage and current of the electromagnetic coil are adjusted by the voltage-and-current control module according to the instruction, thereby controlling the magnitude of the electromagnetic force. In this way, the deformation degree of the flexible component 4 can be controlled to generate the desired suction force level, and the desired suction force level is fed back to the display module for display.

After turning on the system, if the power button is pressed again, the breast pump will shut down.

In an embodiment, the button operation instruction is a mode switching instruction, a shift-up instruction, a shift-down instruction or a pause instruction.

In this embodiment, the mode switching instruction, the shift-up instruction, the shift-down instruction and the pause instruction respectively correspond to the mode switch button, the “+” button, the “−” button and the pause button on the breast pump. Pressing a corresponding button can issue a corresponding instruction.

In an embodiment, after the breast pump starts working, the method further includes the following step. Whether a voltage of the breast pump is lower than a minimum working voltage is determined by the power detection module. If yes, the breast pump is controlled to automatically shut down. Otherwise, the breast pump is controlled to continue to work.

In this embodiment, the minimum working voltage is 3.3 V. If the voltage is lower than 3.3 V, the breast pump is controlled to automatically shut down. If the voltage is higher than 3.3 V, the breast pump is controlled to continue to work.

In an embodiment, after the breast pump starts working, the method further includes the following step. Whether the breast pump continuously works for a preset time is determined by the MCU control module. If yes, the breast pump is controlled to automatically shut down. Otherwise, the breast pump is controlled to continue to work.

In this embodiment, the preset time is 20 min. If the breast pump continuously works for 20 min, the breast pump is controlled to automatically shut down. If the breast pump continuously works for less than 20 min, the breast pump is controlled to continue to work.

The embodiments described above are merely illustrative of the present disclosure, and are not intended to limit the patent scope of the present in disclosure. Any equivalent structural transformation or direct/indirect application in other related technical fields made using the description and drawings of the present disclosure without departing from the concept of the disclosure shall fall within the scope of the disclosure defined by the appended claims.

What is claimed is:

1. A breast pump control method, comprising:
step (1) providing a breast shield with a first cavity, and allowing the breast shield to fit and at least partially accommodate a breast to form a sealed area;
step (2) providing a flexible component, wherein the flexible component is provided with a first magnetic component; and communicating at least a portion of the flexible component with the sealed area to form a portion of a wall of the sealed area; and
step (3) arranging a second magnetic component opposite to the first magnetic component; and driving, by the second magnetic component, the first magnetic component to move to drive the flexible component to deform, such that a negative pressure is generated in the sealed area to express milk from the breast.

2. The breast pump control method of claim 1, wherein the step (3) further comprises:
controlling the second magnetic component to switch between a S-pole and an N-pole, such that the flexible component is driven by the first magnetic component to perform reciprocating motion.

3. The breast pump control method of claim 1, wherein the step (1) further comprises:
connecting a connector to the breast shield; wherein the connector has a hollow structure; and an end of the connector is provided with an opening, and is connected to a converging end of the breast shield;
arranging a mounting structure at the connector; wherein an interior of the mounting structure is communicated with the connector; and
arranging the flexible component in the mounting structure, such that the breast shield and the flexible component form the sealed area through the connector.

4. The breast pump control method of claim 3, wherein the step (3) further comprises:
fixing the second magnetic component in a housing, and connecting the second magnetic component to a control mechanism; wherein the control mechanism is arranged in the housing.

5. The breast pump control method of claim 4, wherein a milk collecting component is connected to the connector.

6. The breast pump control method of claim 5, wherein the milk collecting component comprises a cover, and the cover and the breast shield are configured to enclose a second cavity for milk collection;
the connector comprises a mounting sleeve, a first connecting tube and a second connecting tube; and
an end of the mounting sleeve is provided with the opening, and is detachably connected to the converging end of the breast shield; a first side of the mounting sleeve is communicated with the mounting structure through the first connecting tube, and a second side of the mounting sleeve is connected to a first end of the second connecting tube to be communicated with the cover.

7. The breast pump control method of claim 6, wherein a second end of the second connecting tube is sleevedly provided with a one-way valve.

8. The breast pump control method of claim 6, wherein an outer edge of the flexible component is configured to be folded along an edge of the mounting structure to form a sealed connection between the outer edge of the flexible component and the mounting structure.

9. The breast pump control method of claim 8, wherein an outer edge of an opening of the mounting structure is provided with a first clamping portion in a hook shape; a bowl wall of the mounting structure and the first clamping portion are configured to together form a hook structure; an upper end of the hook structure is configured to be inclined toward a bottom of the cover; the outer edge of the flexible component is configured to bend downward to form an annular groove; the outer edge of the flexible component is provided with a second clamping portion in a hook shape; the second clamping portion is configured to extend to an interior of the annular groove, and is configured to be in snap-fit with the first clamping portion when the first clamping portion is inserted into the annular groove.

10. The breast pump control method of claim 6, wherein an inner wall of the cover is provided with a convex ring for abutting against the flexible component.

11. The breast pump control method of claim 10, wherein the cover is connected to the mounting structure through the convex ring; the convex ring extends from an opening of the mounting structure into the mounting structure; and the flexible component is configured to be clamped by the convex ring and the mounting structure.

12. The breast pump control method of claim 6, wherein an outer wall of the cover is provided with a mounting portion, and the housing is detachably mounted on the mounting portion.

13. The breast pump control method of claim 6, wherein the housing is provided with a plurality of buttons, and the plurality of buttons are at least configured to control deformation of the flexible component under an action of a magnetic force.

14. A breast pump circuit control system applied to the breast pump control method of claim 1 for circuit control, comprising:
a microcontroller unit (MCU) control module;
an interactive control module; and
a voltage-and-current control module;
wherein the MCU control module is configured for signal reception and transmission;
the interactive control module is configured to receive an operation signal from a user and transmit the operation signal to the MCU control module; and
the voltage-and-current control module is configured to receive a control signal transmitted by the MCU control module, control a voltage and a current passing through an electromagnetic coil to reach a desired suction force level, and transmit a suction level signal back to the MCU control module.

15. The breast pump circuit control system of claim 14, further comprising:
a power detection module;
a charging detection module; and
a display module;
wherein the power detection module is configured to monitor a power level of a power supply device in real time and transmit the power level to the MCU control module;
the charging detection module is configured to detect an external charger and feed a detection result back to the MCU control module;
the MCU control module is configured to receive signals from the voltage-and-current control module, the power detection module and the charging detection module, and transmit the signals to the display module; and
the display module is configured to receive and display the signals transmitted by the MCU control module.

16. A breast pump circuit control method for controlling the breast pump circuit control system of claim 14, comprising:
(1) determining, by the interactive control module, whether there is a button operation instruction; if yes, transmitting the button operation instruction to the MCU control module; otherwise, controlling a breast pump to automatically shut down;
(2) receiving and judging, by the MCU control module, the button operation instruction transmitted by the interactive control module followed by transmitting the button operation instruction to the voltage-and-current control module; and
(3) after receiving the button operation instruction, controlling, by the voltage-and-current control module, the voltage and the current passing through the electromagnetic coil, thereby controlling a magnitude of an electromagnetic force to generate the desired suction force level.

17. The breast pump circuit control method of claim 16, wherein the button operation instruction is a mode switching instruction, a shift-up instruction, a shift-down instruction or a pause instruction.

18. The breast pump circuit control method of claim 16, further comprising:
before step (1), determining, by the charging detection module, whether an external charger is connected; if yes, transmitting a charging status signal to the display module for display.

19. The breast pump circuit control method of claim 16, further comprising:
determining, by the power detection module, whether a voltage of the breast pump is lower than a minimum working voltage; if yes, controlling the breast pump to automatically shut down; otherwise, controlling the breast pump to continue to work;
determining, by the MCU control module, whether the breast pump continuously works for a preset time; if yes, controlling the breast pump to automatically shut down;
otherwise, controlling the breast pump to continue to work;
after the interactive control module determines that there is the button operation instruction, pressing a power button to turn on the breast pump; and
after the breast pump is turned on, pressing the power button again to turn off the breast pump.
